(19) United States
(12) Patent Application Publication
(10) Pub. No.: US 2025/0160873 A1
(43) Pub. Date: May 22, 2025

(54) MEDICAL DEVICES WITH RE-JOINABLE HOUSINGS

(71) Applicant: Stryker Corporation, Portage, MI (US)

(72) Inventors: Raymond Michael WOLNIEWICZ, III, Scottsdale, AZ (US); Jason Gregory SEIGER, Gilbert, AZ (US); Nathan Rampton BUTTERS, Phoenix, AZ (US); Nicholas Anthony Chesley FERENTHEIL, Gilbert, AZ (US)

(73) Assignee: Stryker Corporation, Portage, MI (US)

(21) Appl. No.: 18/948,914

(22) Filed: Nov. 15, 2024

Related U.S. Application Data
(60) Provisional application No. 63/600,627, filed on Nov. 17, 2023.

(51) Int. Cl. A61B 17/32; A61B 17/00

(52) U.S. Cl. CPC A61B 17/320068; A61B 2017/00477; A61B 2017/00526

(57) ABSTRACT

A medical device includes at least two housing portions joined together at at least one sacrificial joint, wherein each housing portion comprises at least one secondary joining portion configured for joining to at least one corresponding secondary joining portion of at least one other housing portion for rejoining the two housing portions together after destructive breaking of the at least one sacrificial joint.

### CROSS-REFERENCE TO RELATED APPLICATIONS

This application claims the benefit of U.S. Provisional Application No. 63/600,627, filed Nov. 17, 2023, the entire contents of which are hereby incorporated by reference herein.

### FIELD

This disclosure is related, generally, to medical devices, and more specifically, to medical device housings.

### BACKGROUND

Many medical devices are designed for limited use (e.g., single use) and are discarded at the end of their marketed lifespan. For example, many harmonic tissue cutters, which use ultrasonic mechanical vibration to cut and seal tissue during surgical procedures, are discarded after each surgical procedure regardless of whether the device is still usable. Despite having short lifespans, limited use medical devices can still be quite expensive. Opportunities exist to lower medical procedure costs by using refurbished medical devices. A refurbished medical device is generally rebuilt to meet safety and performance requirements that are comparable to its condition when new. A typical refurbishment process includes disassembly, cleaning and disinfection, mechanical and electrical refurbishment, reassembly, and quality testing and labeling. Limited use medical devices are often not designed to be disassembled and reassembled. For example, plastic housings may be used that require destructive disassembly. Replacement of such housings would drive up refurbishment costs, reducing the cost reduction benefit of refurbishing limited use medical devices.

### SUMMARY

According to an aspect, described herein are medical devices that include housings that are configured to facilitate refurbishment by including joints that can be broken and re-made. A medical device can have a housing formed from two housing portions that are joined together at a joint that is formed at initial manufacturing by a process, such as adhesive bonding, mechanical joining, or welding, that does not permit the housing portions to be separated without some destruction of the joint or housing portions. During a refurbishment process, the housing portions are separated from one another by breaking at the joint or at a breaking feature designed into one or both of the housing portions. Separation of the housing portions may enable components housed within to be cleaned, sterilized, repaired, refurbished, and/or replaced and/or may permit disassembly of components needing repair or replacement. The housing portions may be rejoined using secondary joining features that are designed into the housing portions. The secondary joining features can include, for example, joining surfaces that remain after features that formed the original joint are broken off or otherwise separated from the housing portions or sacrificial material that is broken off and replaced. Housings configured according to the principles described herein can enable more cost-effective refurbishment of medical devices.

According to an aspect, a medical device includes at least two housing portions joined together at at least one sacrificial joint, wherein each housing portion comprises at least one secondary joining portion configured for joining to at least one corresponding secondary joining portion of at least one other housing portion for rejoining the two housing portions together after destructive breaking of the at least one sacrificial joint.

The at least one sacrificial joint may include at least one primary joining portion of each of the at least two housing portions, wherein the at least one primary joining portion of a housing portion is bonded to a corresponding primary joining portion of at least one other housing portion. The at least one secondary joining portion may meet the at least one primary joining portion at at least one separation portion. The at least one separation portion may be configured so that the at least one primary joining portion can be removed upon disassembly of the at least two housing portions during reworking of the medical device, and the at least one secondary joining portion of a housing portion can be joined to the at least one corresponding secondary joining portion of at least one other housing portion to rejoin the at least two housing portions together. A volume of an enclosure defined by the at least two housing portions joined together may be less when the primary joining portions are joined together than when the secondary joining portions are joined together. The at least one primary joining portion and the at least one secondary joining portion may each include a flange, the flange comprising a joining surface for joining to a corresponding joining surface of a corresponding flange of at least one other housing portion. The joining surfaces of the primary and secondary joining portions may be the same size. A plurality of primary joining portions may be interleaved with a plurality of secondary joining portions. The plurality of secondary joining portions of one of the at least two housing portions may contact the plurality of secondary joining portions of another of the at least two housing portions when the primary joining portions are joined. The at least one primary joining portion of the at least two housing portions may be bonded together by welding or adhesive.

The at least one secondary joining portion may include a portion of an outer surface of the medical device when the at least two housing portions are joined together at the at least one sacrificial joint. The at least one secondary joining portion may be located within an outer surface of the of the medical device when the at least two housing portions are joined together at the at least one sacrificial joint. The at least one secondary joining portions of the at least two housing portions may be configured for being bonded together for rejoining the at least two housing portions together.

The at least one sacrificial joint may include at least one sacrificial portion configured to be removed prior to joining the secondary joining portions together. The at least one sacrificial portion may be configured to be removed by dissolving with a solvent. The secondary joining portions may be bonded to the at least one sacrificial portion. The secondary joining portions may be configured for being bonded to at least one replacement sacrificial portion for rejoining the at least two housing portions together.

The medical device may be a handheld device. The medical device may include a handle that is formed at least in part by at least a portion of the at least two housing portions.

According to an aspect, a method for reworking a medical device includes breaking at least one sacrificial joint between at least two housing portions of the medical device; and rejoining the at least two housing portions by joining together secondary joining portions of the at least two housing portions.

The at least one sacrificial joint may include respective primary joining portions of the at least two housing portions and the respective primary joining portions may be removed prior to rejoining the at least two housing portions. Removing the respective primary joining portions of the at least two housing portions may include dissolving the primary joining portions with a solvent. Removing the respective primary joining portions of the at least two housing portions may include breaking the respective primary joining portions off from the at least two housing portions at preformed breaking locations. The respective primary joining portions of the at least two housing portions may be broken off from the secondary joining portions.

Breaking the at least one sacrificial joint between the at least two housing portions may include removing at least one sacrificial portion that was bonded to the secondary joining portions. Rejoining the at least two housing portions by joining together the secondary joining portions may include bonding the secondary joining portions to at least one replacement sacrificial portion.

Rejoining the at least two housing portions by joining together the secondary joining portions may include bonding the secondary joining portions directly together. The at least two housing portions may meet at a plurality of discrete meeting portions, wherein the at least one sacrificial joint may include a first set of the discrete meeting portions that are bonded together and the secondary joining portions may include a second set of the discrete meeting portions that are not bonded together. Joining together the secondary joining portions may include bonding at least a portion of the second set of the discrete meeting portions together.

It will be appreciated that any of the variations, aspects, features, and options described in view of the systems apply equally to the methods and vice versa. It will also be clear that any one or more of the above variations, aspects, features, and options can be combined.

### BRIEF DESCRIPTION OF THE FIGURES

The invention will now be described, by way of example only, with reference to the accompanying drawings, in which:

FIG. 1 illustrates an example of a medical device configured for enabling rejoining of housing portions after disconnection of the housing portions during refurbishment;

FIGS. 2A and 2B show an example of the destructive separation of housing portions;

FIGS. 3A-3E are cross-sectional views of exemplary housing portions that are configured for rejoining after destructive separation;

FIGS. 4A-4D illustrate an example of housing portions that can be rejoined using sacrificial material;

FIG. 5 illustrates an example of housing portions that include interleaved joining portions configured for rejoining after destructive separation;

FIGS. 6A and 6B illustrate an example of re-joinable housing portions in which the secondary joining portions are located behind the primary joining portions so that the secondary joining portions are contained within the housing when the housing portions are initially joined together;

FIGS. 6C and 6D illustrate examples of re-joinable housing portions in which the joining portions are pins (FIG. 6C) or pins and receptacles (FIG. 6D); and

FIG. 7 illustrates an example of the rejoining of housing portions in which material damaged when separating the housing portions is removed in a manner that leaves behind surfaces that are suitable for rejoining.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Described herein are housings for medical devices in which secondary joining features are designed into the housings to facilitate refurbishment of the medical devices. Many medical devices, such as hand-held surgical tools, utilize two or more outer housing portions to protect components housed within during use. To prevent unwanted separation of these housing portions during use, joining methods may be used that make separation of the housing portions difficult. Examples of such joining methods include adhesively bonding the housing portions, ultrasonically welding the housing portions together, using press fit pins, and using locking features, such as locking tabs and snap lock pins. Separation of the housing portions joined with such methods typically result in damage to one or both housing portions. The housing portions described herein include secondary joining portions that allow for rejoining of the housing portions after such damage. Thus, housings can be made using joining methods that prevent unwanted separation while also allowing ease of separation after use. Medical devices including such housings can be refurbished without having to replace the housings, which provides for less costly refurbishment leading to lower cost medical devices and, thereby, lower cost medical procedures and further reduced medical waste.

Housing portions may include primary joining portions that include breaking/fracturing features or sacrificial material at a joint between the two housing portions that are inherently weaker than surrounding material, allowing the joint to be broken apart and the housing portions separated. The broken portions of the primary joining portions can be removed, leaving secondary joining portions that can be joined together for subsequent joining of the housing portions. Such features could be included at the perimeter of outer housing portions and/or can be included in internal joining features, such as internal pins.

In the following description of the various embodiments, reference is made to the accompanying drawings, in which are shown, by way of illustration, specific embodiments that can be practiced. It is to be understood that other embodiments and examples can be practiced and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain terminology is used in this description for convenience and reference only and is not limiting. For example, the words “upwardly,” “downwardly,” “rightwardly,” and “leftwardly” refer to directions in the drawings to which reference is made. The words “inwardly” and “outwardly” refer to directions toward and away from, respectively, the geometric center of the arrangement and designated parts thereof. The words “forwardly” and “distally” refer to the direction toward the end of the arrangement that is closest to the subject, and the words “rearwardly” and “proximally” refer to the direction toward the end of the arrangement which is furthest from the subject. This terminology includes the words specifically mentioned, derivatives thereof, and words of a similar nature.

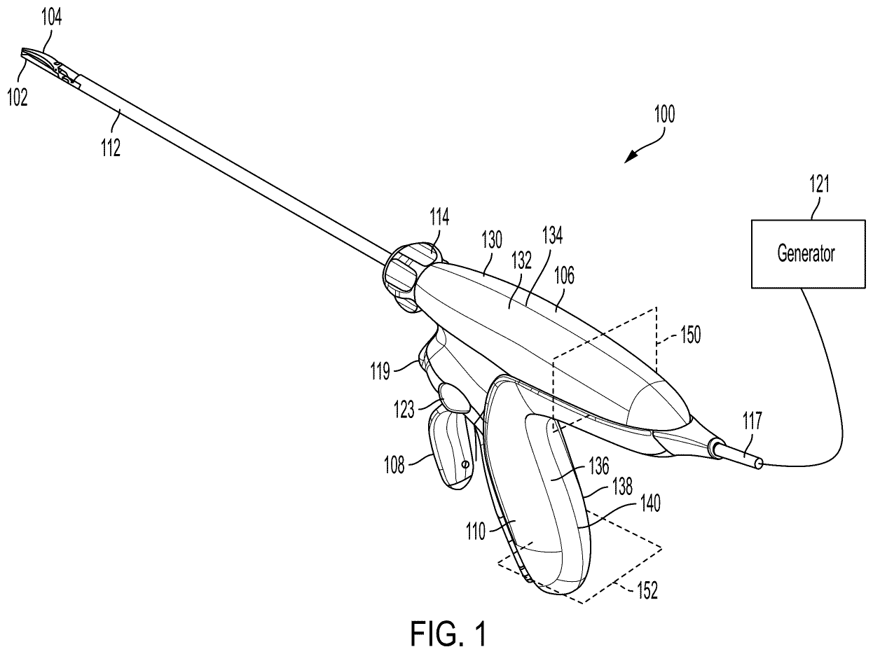

FIG. 1 illustrates an example of a medical device configured for enabling rejoining of housing portions after disconnection of the housing portions during refurbishment. The example of FIG. 1 is a harmonic tissue cutter 100, which is a handheld surgical tool used to cut and/or seal tissue during any suitable surgical procedure, including open field procedures and minimally invasive procedures. The harmonic tissue cutter 100 includes a blade 102 and a jaw 104 that can pivot for grasping tissue between the jaw 104 and the blade 102. The blade 102 is mounted to a shaft (not shown) that extends into the main body 106. A harmonic transducer (not shown) within the main body 106 drives the shaft harmonically, causing the blade 102 to vibrate. The vibration of blade 102 can cut and seal tissue grasped between the blade 102 and jaw 104. The harmonic tissue cutter 100 may be connected via a cable 117 to a harmonic generator 121 that generates the energy to drive the transducer. A user may press an activation button 119 that interacts with one or more switches (not shown) within the main body 106 to activate the harmonic transducer. A selector button 123 can be used to select an amount of vibration of the blade 102. The jaw 104 is opened and closed via a lever 108 of the handle assembly 110. The user can squeeze the lever 108 while gripping the handle assembly 110 to close the jaw 104 and put pressure on tissue grasped between the jaw 104 and the blade 102. The jaw 104 is pivotably mounted to a shaft assembly 112 that extends from the main body 106. A knob 114 may be provided to enable the user to rotate the shaft assembly 112, jaw 104, and blade 102.

The usable lifespan of the harmonic tissue cutter 100 may be limited due to wear of one or more of its components. The lifespan of the harmonic tissue cutter 100 can be increased by repairing or replacing worn components or components that are at or near an end of their usable life. For example, various movable components, such as the selector button 123, the activation button 119, one or more switches within the main body 106 that interface with the selector button 123 or activation button 119, and/or the harmonic transducer, may wear over time and may be replaced to extend the life of the harmonic tissue cutter 100. Additionally or alternatively to being replaced, various components may be cleaned, sterilized, repaired, and/or refurbished to extend the life of the harmonic tissue cutter 100. Such refurbishment may include opening up the housing(s) of the main body 106 and/or handle assembly 110 to access components within for cleaning, sterilization, repair, and/or replacement and/or to enable disassembly of components attached thereto, such as the shaft assembly 112. Opening up the housing(s) of the main body 106 and/or handle assembly 110 may require separating housing portions from each other to access the components within. For example, the main body 106 may include housing portion 130 and housing portion 132 and opening up the housing of the main body 106 may include separating housing portion 130 from housing portion 132. Similarly, the handle assembly 110 may include housing portion 136 and housing portion 138, and opening up the housing of the handle assembly 110 may include separating housing portion 136 from housing portion 138. The housing portions 130, 132, and/or housing portions 136, 138 may be joined together at least in part at respective joints 134, 140 that are formed in a non-separable manner, such as by using adhesive, ultrasonic welding, or another process that joins the housing portions 130, 132 together in a manner that would require some destruction to separate the housing portions 130, 132 from each other. Such a joint may be less costly to manufacture than separable joint types, such as joints that use fasteners, and may provide a higher level of tamper resistance than separable joints. As a result, refurbishment that requires separating the housing portions 130, 132 may require the joints 134, 140 to be destructively separated. According to the principles described within, housing components can be configured to enable rejoining after destruction of the joints between the housing components. This enables refurbishment of medical devices, such as surgical tools, without requiring the housing components to be replaced, which reduces the cost of refurbishment.

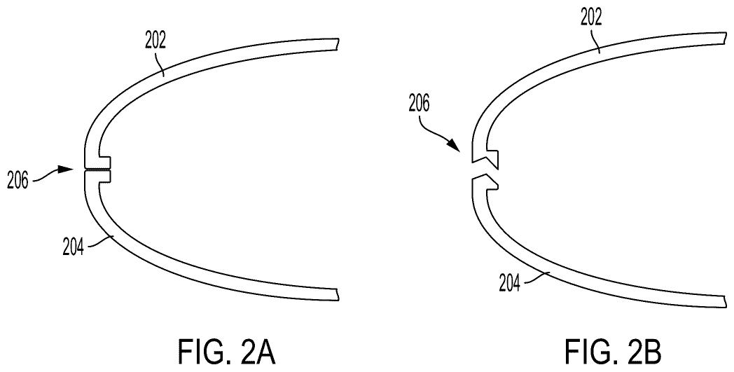

FIGS. 2A and 2B illustrate the destructive separation of two conventional housing portions 202, 204 that were joined together at a non-separable joint 206. As used herein, “non-separable” means that that the components are not intended to be separated and rejoined without further processing. Non-separable joints can include adhesive joints, welded joints (e.g., hot gas welded, laser welded, ultrasonic welded, solvent welded, or hot plate welded), mechanical joints that require breakage or deformation for separation (e.g., tabs received in slots), or any other joining method that requires breakage or deformation for separation. FIG. 2A shows the joint 206 before separation and FIG. 2B shows the joint after the joint 206 has been broken. Breaking of the joint 206 can leave a jagged, broken surface that may not be suitable for rejoining, or even if re-joinable, may result in an unacceptable visual appearance.

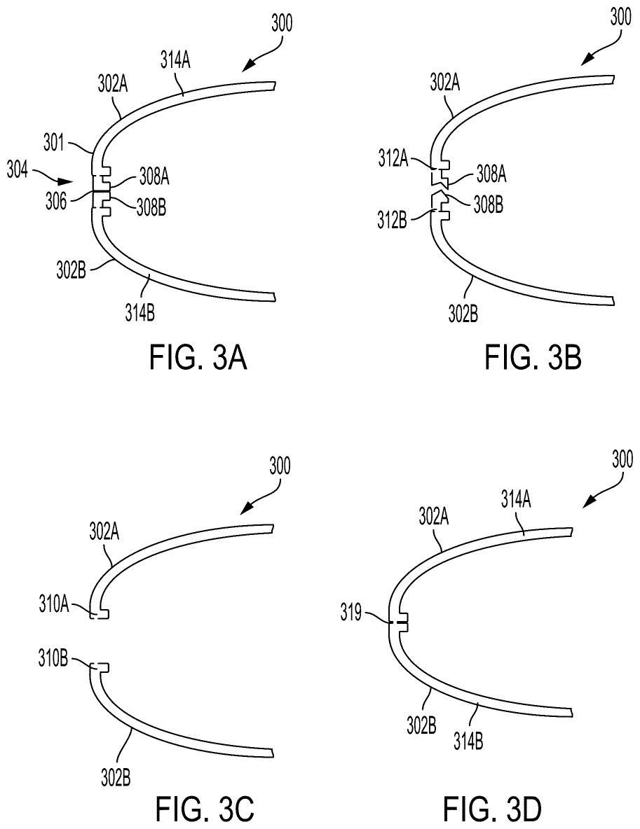

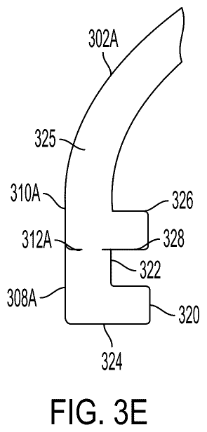

FIGS. 3A-D are cross-sectional views of exemplary housing portions 302A, 302B of a surgical tool that are configured for rejoining after being destructively separated, according to an aspect of the present disclosure. The housing portions 302A, 302B may be used for any housing of a surgical tool, such as for housing portions 130, 132 of main body 106 and/or housing portions 136, 138 of handle assembly 110 of FIG. 1. For example, FIG. 3A can represent a cross-sectional view of housing portions 130, 132 taken with respect to plane 150 of FIG. 1 and/or a cross-sectional view of housing portions 136, 138 taken with respect to plane 152 of FIG. 1. The housing portions 302A, 302B are joined together during original manufacture at a sacrificial joint 304 (which is an example of joint 134 of FIG. 1), forming a housing 300 as shown in FIG. 3A. The sacrificial joint 304 may include a seam 306 where respective joining portions 308A, 308B of the two housing portions 302A, 302B have been bonded together using a non-separable bonding process, such as an adhesive bonding or welding. For example, the two housing portions 302A, 302B may be made of a plastic (e.g., ABS, polycarbonate, polypropylene or polyamide) and may have been bonded to together using adhesive or any suitable plastic welding process (e.g., hot gas welded, laser welded, ultrasonic welded, solvent welded, or hot plate welded). Optionally, the housing portions 302A, 302B can be originally formed together as a single piece (e.g., molded as a single piece) in which the sacrificial joint 304 is a weakened area formed into the single piece so that the two housing portions 302A, 302B can be broken apart at the weakened area.

The housing portions 302A, 302B may be separated to access components housed within during refurbishment of the surgical tool by destructively breaking the housing portions 302A, 302B apart, the results of which are illustrated in FIG. 3B. Breakage, which may occur at least in part along seam 306, may leave damaged surfaces that are unsuitable for rejoining, as discussed above with respect to FIGS. 2A and 2B. To facilitate rejoining, housing portions 302A, 302B may each include secondary joining portions 310A, 310B (FIG. 3C). The primary joining portions 308A, 308B and the secondary joining portions 310A, 310B may form portions of an outer surface 301 of the housing 300 formed by housing portions 302A, 302B.

Each secondary joining portion 310A, 310B meets the respective primary joining portion 308A, 308B at a respective separation portion 312A, 312B. The separation portions 312A, 312B may be regions extending on either side of the seam 306 and spaced from the seam 306 that are configured so that the primary joining portions 308A, 308B may be separated (e.g., broken off) from the housing portions 302A, 302B, leaving the secondary joining portions 310A, 310B. As a result, the primary joining portions 308A, 308B may be sacrificial portions that are broken off and discarded. The separation portions 312A, 312B may be weaker than surrounding regions to provide for preferential breakage at the separation portions 312A, 312B. For example, relative to surrounding regions, the separation portions 312A, 312B may include reduced material thickness, may be formed of a weaker material, may include stress risers, or may include indentations, perforations, or other features that weaken the separation portions 312A, 312B relative to surrounding regions. The primary joining portions 308A, 308B may be removed in any suitable way, including by breaking them off as mentioned above, by machining them off, by dissolving them with a solvent, or by any combination of these methods or by using any other suitable technique for removing the primary joining portions 308A, 308B. Optionally, the separation portions 312A, 312B may be interfaces between two different surfaces that were bonded together. For example, the primary joining portions 308A, 308B (e.g., the sacrificial portions) may be bonded to the secondary joining portions 310A, 310B at the separation portions 312A, 312B. Such a bond may be dissolved with a solvent or otherwise weakened in order to separate the primary joining portions 308A, 308B from the secondary joining portions 310A, 310B.

After the housing portions 302A, 302B have been broken apart from one another (FIG. 3B), the remnants of the primary joining portions 308A, 308B may be broken off at the separation portions 312A, 312B, leaving behind the secondary joining portions 310A, 310B, as shown in FIG. 3C. The secondary joining portions 310A, 310B may then be bonded to one another to rejoin the housing portions 302A, 302B together at a reformed joint 319, reassembling the housing 300 (as shown in FIG. 3D). The secondary joining portions 310A, 310B may be bonded using the same bonding process used for bonding the primary joining portions 308A, 308B (e.g., welding or adhesive) or a different bonding process.

Because the primary joining portions 308A, 308B are broken off in order to bond the secondary joining portions 310A, 310B, the rejoining process may result in a decrease of the separation of walls 314A, 314B of the housing portions 302A, 302B, as shown by the difference between distance D₁ of FIG. 3A and distance D₂ of FIG. 3D. Thus, the rejoined housing portions 302A, 302B may enclose a smaller volume than the originally joined housing portions 302A, 302B. Nevertheless, the housing portions 302A, 302B are configured to house components within and/or assemble with other features of the surgical tool in both the original joined state and the rejoined state.

FIG. 3E is an enlarged view of a portion of housing portion 302A. Housing portion 302B may be similarly configured and, thus, is not described separately here. The primary joining portion 308A may include a flange 320 extending from a wall 322 that extends from the secondary joining portion 310A. The flange 320 provides a joining surface 324 for bonding to a corresponding bonding surface of the housing portion 302B. The secondary joining portion 310A may have a flange 326 to which the wall 322 is connected at the separation portion 312A. Once the primary joining portion 308A is broken off from the flange 326, the flange 326 provides a joining surface 328. The flange 326 of the secondary joining portion 310A may have the same shape as the flange 320 of the primary joining portion 308A so that the secondary joining portion 310A provides a similar joining strength as the primary joining portion 308A. As a result, joining surfaces 324, 328 may be the same size. Alternatively, the flanges 320, 326 may have different shapes. The wall 322 of the primary joining portion 308A may have the same thickness as a main wall 325 of the housing portion 302A or may be thicker or thinner.

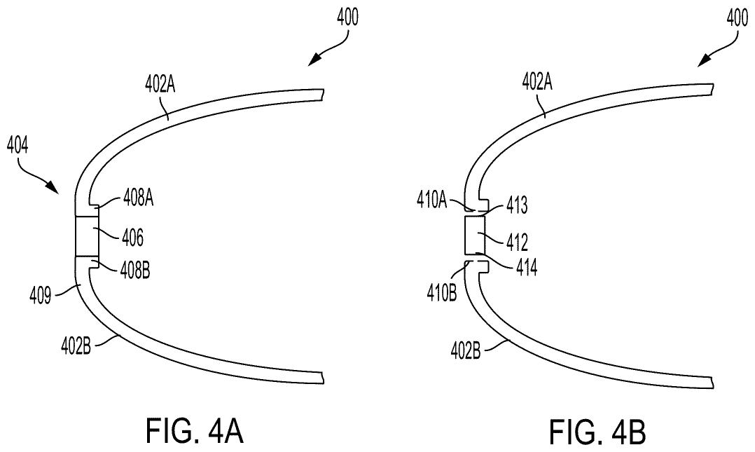

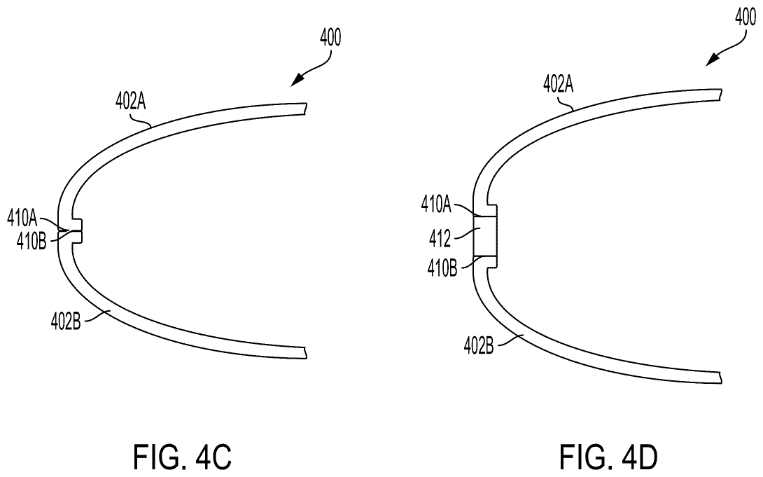

FIGS. 4A-D illustrate another example of housing portions configured for rejoining after being destructively separated. The housing portions of FIGS. 4A-D may be used for any housing of a surgical tool, such as for housing portions 130, 132 of main body 106 and/or housing portions 136, 138 of handle assembly 110 of FIG. 1. Referring first to FIG. 4A, the housing 400 is formed by a first housing portion 402A connected to a second housing portion 402B at a sacrificial joint 404. The sacrificial joint 404 includes a sacrificial component 406 that is joined at each end to secondary joining portions 408A, 408B of the housing portions 402A, 402B. The sacrificial component 406 may form a portion of an outer surface 409 of the housing 400. A first side 413 of the sacrificial component 406 is joined (e.g., adhesively bonded or welded) to the secondary joining portion 408A of the first housing portion 402A and a second side 414 of the sacrificial component 406 is joined (e.g., adhesively bonded or welded) to the secondary joining portion 408B of the second housing portion 402B. The housing portions 402A, 402B can be separated (e.g., during refurbishment) by breaking the bond between the sacrificial component 406 and one or both of the secondary joining portions 408A, 408B. The sacrificial component 406 may be separated from both secondary joining portions 408A, 408B (as shown in FIG. 4B) and discarded. Separation of the sacrificial component 406 from the secondary joining portions 408A, 408B exposes surfaces 410A, 410B that can be used for rejoining the housing portions 402A, 402B. The surfaces 410A, 410B may be prepared for rejoining, such as by using an abrasive material or machining to smooth out the surfaces 410A, 410B. The surfaces 410A, 410B may then be bonded directly together to rejoin the housing portions 402A, 402B, as shown in FIG. 4C, or may be bonded to a replacement sacrificial component 412, as shown in FIG. 4D. Rejoining without the replacement sacrificial component 412 may reduce the volume of the enclosure provided by the housing 400. Rejoining with the replacement sacrificial component 412 can result in the same volume of the enclosure provided by the housing 400. The sacrificial component 412 can be used in the examples of FIGS. 3A-E by bonding the joining surfaces 324 of secondary joining portions 310A, 310B to the sacrificial component 412 instead of to each other. In such examples, the sacrificial component 412 can be sized similarly to the combined sizes of the primary joining portions 308A, 308B so that the rejoined housing provides the same enclosed volume as the originally joined housing.

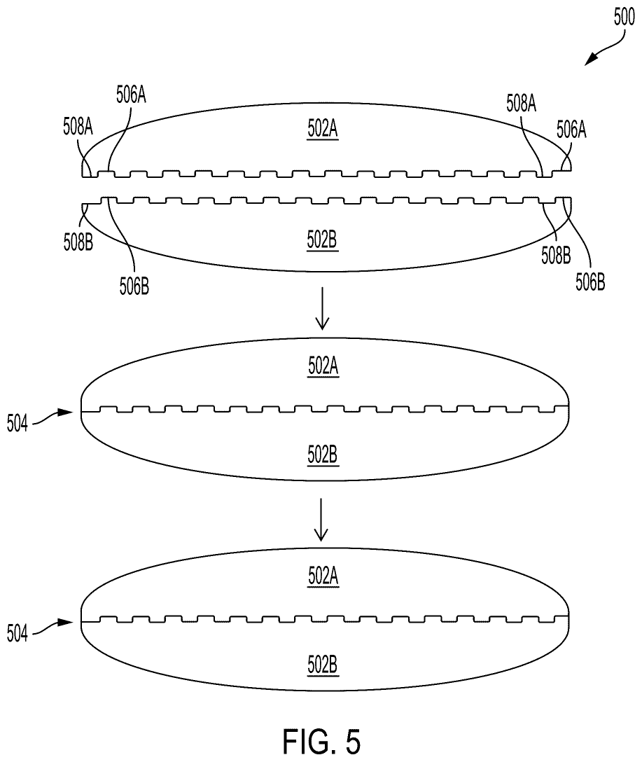

FIG. 5 illustrates another example of housing portions configured for rejoining after being destructively separated, which can be used for a surgical tool, such as harmonic tissue cutter 100 of FIG. 1. In the illustrated example, two housing portions 502A, 502B are joined together at a joint 504 which is formed by a plurality of discrete joining portions, which may interlock together. A set of the joining portions are bonded together at initial manufacture (e.g., welded or adhesively bonded together) and one or more other sets of joining portions are not. The joining portions that are bonded together at initial manufacture are referred to as primary joining portions and include primary joining portion 506A of housing portion 502A and primary joining portion 506B of housing portion 502B. The joining portions that are not bonded together at initial manufacture are referred to as secondary joining portions (e.g., secondary joining portion 508A of housing portion 502A and secondary joining portion 508B of jousting portion 502B. The secondary joining portions may contact one another at initial manufacture but may not be bonded.

The primary joining portions are destructively separated from each other to separate the housing portions 502A, 502B. To rejoin the housing portions 502A, 502B, the secondary joining portions are bonded together (e.g., welded or adhesively bonded together). The primary joining portions may not be removed from their respective housing portions. However, when rejoining the housing portions 502A, 502B, the primary joining portions 506A, 506B may not be joined together since they may not be suitable for rejoining due to the destructive separation process.

The housing portions 502A, 502B may include secondary joining portions that are not bonded together during rejoining. These may be used for subsequent rejoining processes (e.g., during a second or further refurbishment). The primary joining portions may be interleaved with the secondary joining portions. For example, the primary joining portions 506A may alternate with the secondary joining portions 508A. In the illustrated example, the primary joining portions are keyed to the secondary joining portions in a gear tooth-like arrangement. However, this is merely exemplary as the primary and secondary joining portions may be in any suitable form.

Although the example illustrated in FIG. 5 includes interlocking joining portions, this is merely exemplary. In some variations, the joining portions (e.g., primary joining portions 506A and secondary joining portions 508A) of a given housing are portions of the same surface. For example, mating surfaces of two housing portions may be intermittently bonded at initial manufacture, and portions that were not bonded at initial manufacture can be bonded during refurbishment to rejoin the housing portions.

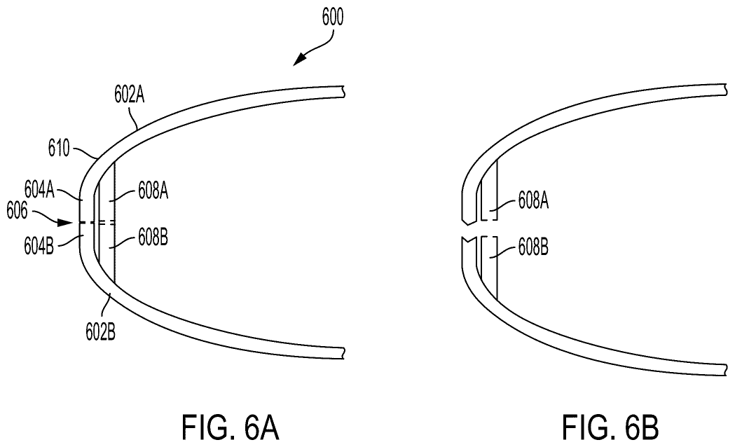

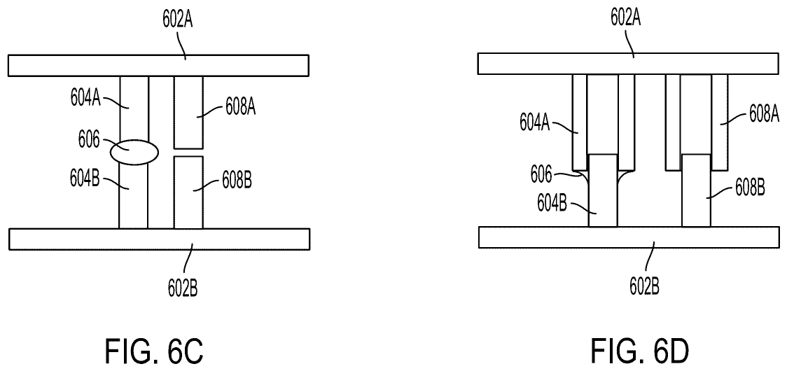

FIGS. 6A and 6B illustrate an example of re-joinable housing portions in which the secondary joining portions are located behind the primary joining portions so that the secondary joining portions are contained within the housing when the housing portions are initially joined together. In the illustrated example, the housing portions 602A, 602B are joined together at a sacrificial joint 606, forming housing 600. As shown in FIG. 6A, joint 606 is initially formed by joining together a primary joining portion 604A of housing portion 602A and a primary joining portion 604B of housing portion 602B, which can be bonded together using any suitable method, including any of the methods described herein. Secondary joining portions 608A, 608B are positioned behind the primary joining portions 604A, 604B. The secondary joining portions 608A, 608B are not initially bonded together. The secondary joining portions 608A, 608B are located within the housing 600—i.e., within the outer surface 610 of the housing 600 when the housing portions 602A, 602B are joined together at the sacrificial joint 606.

The primary joining portions 604A, 604B are destructively separated to separate the housing portions 602A, 602B for refurbishment of the surgical tool. The interface between the primary joining portions 604A, 604B may be broken to separate the primary joining portions 604A, 604B. Alternatively, one or both of the primary joining portions 604A, 604B may include a breaking portion configured to break when a separation force is applied to the housing portions 602A, 602B. Remnants of the primary joining portions 604A, 604B may be removed after the housings 602A, 602B have been separated. The secondary joining portions 608A, 608B can then be bonded together to rejoin the housing portions 602A, 602B together. The secondary joining portions 608A, 608B may be configured so that the housing 600 has the same shape (e.g., the same enclosed volume) as it had when the primary joining portions 604A, 604B were joined together.

The secondary joining portions 608A, 608B may be similar in configuration to the primary joining portions 604A, 604B. For example, wherein the primary joining portions 604A, 604B include a wall from point A to point B, the secondary joining portions 608A, 608B may include a wall from point A to point B (behind the wall of the primary joining portions). The secondary joining portions 608A, 608B may have a different configuration than the primary joining portions 604A, 604B. For example, the primary joining portions 604A, 604B may be a wall and the secondary joining portions 608A, 608B may be pins or a series of pins.

Although primary joining portions 604A, 604B are shown as portions of an outer wall of the housing 600, this is merely exemplary. The primary joining portions 604A, 604B can be features (e.g., walls, struts, or pins) internal to the outer wall of the housing 600. For example, the primary joining portions 604A, 604B can include pins located within the housing 600 (i.e., extending inwardly from an inner surface of the housing 600). Examples of this are illustrated in FIGS. 6C and 6D. In the example of FIG. 6C, the primary joining portions 604A, 604B are pins that are bonded to each other at the sacrificial joint 606, and the secondary joining portions 608A, 608B are also pins that are not bonded upon initial manufacture but that can be bonded (e.g., adhesively attached or welded) during refurbishment. In the example illustrated in FIG. 6D, primary joining portion 604A is a receptacle that receives primary joining portion 604B, which is in the form of a pin. The two are bonded together at sacrificial joint 606. The secondary joining portions 608A and 608B are a corresponding receptacle and pin, respectively. The secondary joining portion 608B may be received in the secondary joining portion 608A (and, thus, may provide some structural support to the housing portions 604A, 602B) but they are not bonded together. Different joining techniques can be used for the primary joining portions 604A, 604B and the secondary joining portions 608A, 608B. For example, the primary joining portions 604A, 604B may be welded and the secondary joining portions 608A, 608B may be adhesively joined or vice versa.

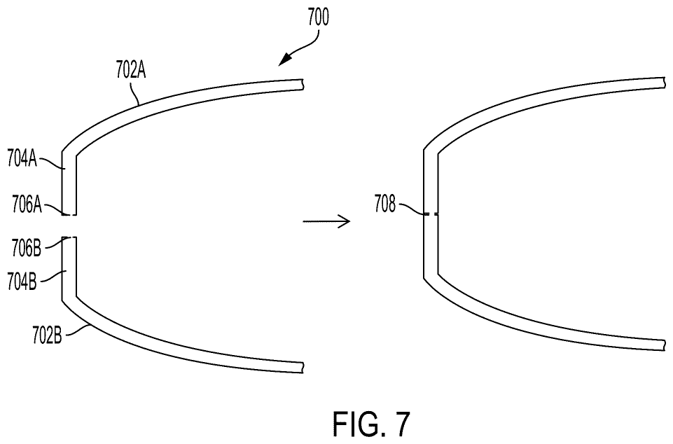

FIG. 7 illustrates the rejoining of housing portions in which material damaged when separating the housing portions is removed in a manner that leaves behind surfaces that are suitable for rejoining. The housing portions 702A, 702B each include joint portions 704A, 704B that are initially joined at a sacrificial joint (not shown) that is broken when the housing portions 702A, 702B are separated for refurbishment of the surgical tool. The damaged portions of the joint portions 704A, 704B can be removed, such as via machining, sanding, grinding, etc., forming surfaces 706A, 706B that are suitable for joining together. To rejoin the housing portions 702A, 702B, the surfaces 706A, 706B are bonded together (e.g., welded or adhesively bonded) into a new joint 708. This may result in a smaller enclosed volume relative to the original enclosed volume. Joint portions 704A, 704B can form portions of an outer wall of the housing 700 and/or can include interior components, such as interior walls or pins.

The figures, as described above, generally show the joining of the housing portions at a single location. It is to be understood that the housing portions may be joined at multiple different locations and that a given joint may extend any suitable distance, may be interrupted at one or more locations, may include multiple discrete sections that are spaced apart from each other, may include changes in shape and/or material thickness, and may include different joint types (including any combination of the joint types described herein). Further, different housing portions of the same surgical device may be joined using different joining techniques, including any combination of the techniques described herein. Other joining techniques may be used in combination with any of the joining techniques described herein. For example, housing portions may be joined by a combination of mechanical fasteners and any of the joining techniques described above.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications that are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

1. A medical device comprising:
at least two housing portions joined together at at least one sacrificial joint,
wherein each housing portion comprises at least one secondary joining portion configured for joining to at least one corresponding secondary joining portion of at least one other housing portion for rejoining the two housing portions together after destructive breaking of the at least one sacrificial joint.

2. The medical device of claim 1, wherein the at least one sacrificial joint comprises at least one primary joining portion of each of the at least two housing portions, wherein the at least one primary joining portion of a housing portion is bonded to a corresponding primary joining portion of at least one other housing portion.

3. The medical device of claim 2, wherein the at least one secondary joining portion meets the at least one primary joining portion at at least one separation portion.

4. The medical device of claim 3, wherein the at least one separation portion is configured so that the at least one primary joining portion can be removed upon disassembly of the at least two housing portions during reworking of the medical device, and the at least one secondary joining portion of a housing portion can be joined to the at least one corresponding secondary joining portion of at least one other housing portion to rejoin the at least two housing portions together.

5. The medical device of claim 2, wherein a volume of an enclosure defined by the at least two housing portions joined together is less when the primary joining portions are joined together than when the secondary joining portions are joined together.

6. The medical device of claim 2, wherein the at least one primary joining portion and the at least one secondary joining portion each comprise a flange, the flange comprising a joining surface for joining to a corresponding joining surface of a corresponding flange of at least one other housing portion.

7. The medical device of claim 6, wherein the joining surfaces of the primary and secondary joining portions are the same size.

8. The medical device of claim 2, wherein a plurality of primary joining portions are interleaved with a plurality of secondary joining portions.

9. The medical device of claim 8, wherein the plurality of secondary joining portions of one of the at least two housing portions contact the plurality of secondary joining portions of another of the at least two housing portions when the primary joining portions are joined.

10. The medical device of claim 2, wherein the at least one primary joining portion of the at least two housing portions are bonded together by welding or adhesive.

11. The medical device of claim 1, wherein the at least one secondary joining portion comprises a portion of an outer surface of the medical device when the at least two housing portions are joined together at the at least one sacrificial joint.

12. The medical device of claim 1, wherein the at least one secondary joining portion is located within an outer surface of the of the medical device when the at least two housing portions are joined together at the at least one sacrificial joint.

13. The medical device of claim 1, wherein the at least one secondary joining portions of the at least two housing portions are configured for being bonded together for rejoining the at least two housing portions together.

14. The medical device of claim 1, wherein the at least one sacrificial joint comprises at least one sacrificial portion configured to be removed prior to joining the secondary joining portions together.

15. The medical device of claim 14, wherein the at least one sacrificial portion is configured to be removed by dissolving with a solvent.

16. The medical device of claim 14, wherein the secondary joining portions are bonded to the at least one sacrificial portion.

17. The medical device of claim 16, wherein the secondary joining portions are configured for being bonded to at least one replacement sacrificial portion for rejoining the at least two housing portions together.

18. The medical device of claim 1, wherein the medical device is a handheld device.

19. The medical device of claim 1, wherein the medical device comprises a handle that is formed at least in part by at least a portion of the at least two housing portions.

20. A method for reworking a medical device comprising:
breaking at least one sacrificial joint between at least two housing portions of the medical device; and
rejoining the at least two housing portions by joining together secondary joining portions of the at least two housing portions.

21. The method of claim 20, wherein the at least one sacrificial joint comprises respective primary joining portions of the at least two housing portions and the respective primary joining portions are removed prior to rejoining the at least two housing portions.

22. The method of claim 21, wherein removing the respective primary joining portions of the at least two housing portions comprises dissolving the primary joining portions with a solvent.

23. The method of claim 21, wherein removing the respective primary joining portions of the at least two housing portions comprises breaking the respective primary joining portions off from the at least two housing portions at preformed breaking locations.

24. The method of claim 21, wherein the respective primary joining portions of the at least two housing portions are broken off from the secondary joining portions.

25. The method of claim 20, wherein breaking the at least one sacrificial joint between the at least two housing portions comprises removing at least one sacrificial portion that was bonded to the secondary joining portions.

26. The method of claim 25, wherein rejoining the at least two housing portions by joining together the secondary joining portions comprises bonding the secondary joining portions to at least one replacement sacrificial portion.

27. The method of claim 25, wherein rejoining the at least two housing portions by joining together the secondary joining portions comprises bonding the secondary joining portions directly together.

28. The method of claim 20, wherein the at least two housing portions meet at a plurality of discrete meeting portions, wherein the at least one sacrificial joint comprises a first set of the discrete meeting portions that are bonded together and the secondary joining portions comprises a second set of the discrete meeting portions that are not bonded together.

29. The method of claim 28, wherein joining together the secondary joining portions comprises bonding at least a portion of the second set of the discrete meeting portions together.
